(19) United States
(12) Patent Application Publication
(10) Pub. No.: US 2023/0135029 A1
(43) Pub. Date: May 4, 2023

(54) COMPOSITIONS AND METHODS FOR INCREASING DIGESTIBILITY OF CALCIUM AND PHOSPHOROUS IN PIGS

(71) Applicant: Premex, Inc., Durham, NC (US)

(72) Inventor: Jorge ESTRADA, Durham, NC (US)

(21) Appl. No.: 17/914,997

(22) PCT Filed: Apr. 8, 2021
(86) PCT No.: PCT/US2021/026355

§ 371 Date: Sep. 27, 2022

Related U.S. Application Data
(60) Provisional application No. 63/007,402, filed on Apr. 9, 2020.

(51) Int. Cl. A61K 31/593; A61P 3/02; A61P 1/14; A23K 20/174; A23K 20/24; A23K 20/26; A23K 50/30

(52) U.S. Cl. CPC A61K 31/593; A61P 3/02; A61P 1/14; A23K 20/174; A23K 20/24; A23K 20/26; A23K 50/30

(57) ABSTRACT

A method of increasing apparent total tract digestibility and retention of calcium and phosphorus in a pig is provided. The method includes administering to the pig an effective amount of a vitamin D composition comprising at least one vitamin D compound. Related compositions are also provided.

### CROSS-REFERENCE TO RELATED APPLICATIONS

This application is a U.S. National Stage Application of PCT/US2021/026355, filed Apr. 8, 2021, which claims the benefit of U.S. Provisional Application No. 63/007,402, filed Apr. 4, 2020, the contents of which are hereby incorporated by reference.

### BACKGROUND

Animals need to absorb and utilize vitamins and minerals in order to maintain healthy physiological stasis. Calcium and phosphorus are some of the most important minerals in an animal's diet. These minerals are vital to the formation of bones and teeth. Calcium is also particularly important for numerous other physiological functions including the regulation of heartbeat. Phosphorus is particularly important for nerve and muscle performance as well as for buffering blood pH. Vitamin and mineral supplements may be taken at regular intervals for optimal absorption and utilization. The interaction of different vitamins and minerals is also an important factor to consider when administering minerals in an animal diet.

### SUMMARY

A method of increasing apparent total tract digestibility and retention of calcium and phosphorus in a pig is provided. The method includes the step of administering to the pig an effective amount of a vitamin D composition comprising at least one vitamin D compound. Upon administration to a pig, the apparent total tract digestibility and retention of calcium and phosphorus is increased. According to one embodiment, the vitamin D composition is concurrently administered with a calcium and phosphorus source. According to one embodiment, the calcium and phosphorus source is dispersed in a feed, feed supplement or a combination thereof. According to one embodiment, the feed, feed supplement or a combination thereof comprises corn and one or more minerals. According to one embodiment, the minerals are selected from the group consisting of vitamin A, vitamin E, vitamin K, thiamin, riboflavin, pyridoxine, vitamin B12, panthothenic acid, niacin, folic acid, biotin, copper, iron, iodine, manganese, sodium selenium, and zinc. According to one embodiment, the vitamin D compound is a vitamin D3 compound. According to one embodiment, the vitamin D3 compound is 1-alpha-hydroxycholecalciferol. According to one embodiment, calcium digestibility is increased by at least about 10%. According to one embodiment, phosphorus digestibility is increased by at least about 5%. According to one embodiment, calcium retention is increased by at least about 110%. According to one embodiment, phosphorous retention is increased by at least about 65%. According to one embodiment, the pig is a sow. According to one embodiment, the sow is in a gestation period.

A composition for increasing the apparent total tract digestibility and retention of calcium and phosphorus in a pig is provided. The composition includes an effective amount of a vitamin D composition comprising at least one vitamin D compound. Upon administration to a pig, the apparent total tract digestibility and retention of calcium and phosphorus is increased. According to one embodiment, the vitamin D composition is concurrently administered with a calcium and phosphorus source. According to one embodiment, the calcium and phosphorus source is dispersed in a feed, feed supplement or a combination thereof. According to one embodiment, the feed, feed supplement or a combination thereof comprises corn and one or more minerals. According to one embodiment, the minerals are selected from the group consisting of vitamin A, vitamin E, vitamin K, thiamin, riboflavin, pyridoxine, vitamin B12, panthothenic acid, niacin, folic acid, biotin, copper, iron, iodine, manganese, sodium selenium, and zinc. According to one embodiment, the vitamin D compound is a vitamin D3 compound. According to one embodiment, the vitamin D3 compound is 1-alpha-hydroxycholecalciferol. According to one embodiment, calcium digestibility is increased by at least about 10%. According to one embodiment, phosphorus digestibility is increased by at least about 5%. According to one embodiment, calcium retention is increased by at least about 110%. According to one embodiment, phosphorous retention is increased by at least about 65%. According to one embodiment, the pig is a sow. According to one embodiment, the sow is in a gestation period.

### DETAILED DESCRIPTION

One or more aspects and embodiments may be incorporated in a different embodiment although not specifically described. That is, all aspects and embodiments can be combined in any way or combination. When referring to the compounds disclosed herein, the following terms have the following meanings unless indicated otherwise. The following definitions are meant to clarify, but not limit, the terms defined. If a particular term used herein is not specifically defined, such term should not be considered indefinite. Rather, terms are used within their accepted meanings.

A method of increasing apparent total tract digestibility and retention of calcium and phosphorus in a pig is provided. The method includes the step of administering to the pig an effective amount of a vitamin D composition comprising at least one vitamin D compound. The vitamin D composition as provided herein may further include one or more inert compounds or carriers that aid in administration. The vitamin D composition as provided herein may be formulated as a liquid, gel, powder, table, capsule, or any other acceptable formulation suitable for administration to pigs.

Upon administration of the vitamin D composition to a pig, the apparent total tract digestibility and retention of calcium and phosphorus is increased. According to one embodiment, the apparent total tract digestibility and retention of calcium and phosphorus is increased regardless of dietary calcium and phosphorus intake. According to one embodiment, the apparent total tract digestibility and retention of calcium and phosphorus is increased in the presence of dietary calcium and phosphorus intake. According to one embodiment, the apparent total tract digestibility and retention of calcium and phosphorus is increased in the presence of dietary calcium and phosphorus intake at a level of 25% of recommended daily intake. According to one embodiment, the apparent total tract digestibility and retention of calcium and phosphorus is increased in the presence of dietary calcium and phosphorus intake at a level of 50% of recommended daily intake. According to one embodiment, the apparent total tract digestibility and retention of calcium and phosphorus is increased in the presence of dietary calcium and phosphorus intake at a level of 75% of recommended daily intake. According to one embodiment, the apparent total tract digestibility and retention of calcium and phosphorus is increased in the presence of dietary calcium and phosphorus intake at a level of 100% of recommended daily intake.

According to one embodiment, the apparent total tract digestibility and retention of calcium and phosphorus is increased within one day of administration of a vitamin D composition. According to one embodiment, the apparent total tract digestibility and retention of calcium and phosphorus is increased within two days of administration of a vitamin D composition. According to one embodiment, the apparent total tract digestibility and retention of calcium and phosphorus is increased within three days of administration of a vitamin D composition. According to one embodiment, the apparent total tract digestibility and retention of calcium and phosphorus is increased within four days of administration of a vitamin D composition. According to one embodiment, the apparent total tract digestibility and retention of calcium and phosphorus is increased within five days of administration of a vitamin D composition. According to one embodiment, the apparent total tract digestibility and retention of calcium and phosphorus is increased within six days of administration of a vitamin D composition. According to one embodiment, the apparent total tract digestibility and retention of calcium and phosphorus is increased within one week of administration of a vitamin D composition.

According to one embodiment, calcium digestibility is increased by at least about 5%. According to one embodiment, calcium digestibility is increased by at least about 10%. According to one embodiment, calcium digestibility is increased by at least about 15%. According to one embodiment, calcium digestibility is increased by at least about 20%. According to one embodiment, calcium digestibility is increased by at least about 25%. According to one embodiment, calcium digestibility is increased by at least about 30%. According to one embodiment, calcium digestibility is increased by at least about 35%. According to one embodiment, calcium digestibility is increased by at least about 40%. According to one embodiment, calcium digestibility is increased by at least about 45%. According to one embodiment, calcium digestibility is increased by at least about 50%. According to one embodiment, calcium digestibility is increased by at least about 55%. According to one embodiment, calcium digestibility is increased by at least about 60%. According to one embodiment, calcium digestibility is increased by at least about 65%.

According to one embodiment, phosphorus digestibility is increased by at least about 5%. According to one embodiment, phosphorus digestibility is increased by at least about 10%. According to one embodiment, phosphorus digestibility is increased by at least about 15%. According to one embodiment, phosphorus digestibility is increased by at least about 20%. According to one embodiment, phosphorus digestibility is increased by at least about 25%. According to one embodiment, phosphorus digestibility is increased by at least about 30%. According to one embodiment, phosphorus digestibility is increased by at least about 35%. According to one embodiment, phosphorus digestibility is increased by at least about 40%. According to one embodiment, phosphorus digestibility is increased by at least about 45%. According to one embodiment, phosphorus digestibility is increased by at least about 50%. According to one embodiment, phosphorus digestibility is increased by at least about 55%. According to one embodiment, phosphorus digestibility is increased by at least about 60%. According to one embodiment, phosphorus digestibility is increased by at least about 65%.

According to one embodiment, calcium retention is increased by at least about 60%. According to one embodiment, calcium retention is increased by at least about 65%. According to one embodiment, calcium retention is increased by at least about 70%. According to one embodiment, calcium retention is increased by at least about 75%. According to one embodiment, calcium retention is increased by at least about 80%. According to one embodiment, calcium retention is increased by at least about 85%. According to one embodiment, calcium retention is increased by at least about 90%. According to one embodiment, calcium retention is increased by at least about 95%. According to one embodiment, calcium retention is increased by at least about 100%. According to one embodiment, calcium retention is increased by at least about 110%.

According to one embodiment, phosphorous retention is increased by at least about 30%. According to one embodiment, phosphorous retention is increased by at least about 35%. According to one embodiment, phosphorous retention is increased by at least about 40%. According to one embodiment, phosphorous retention is increased by at least about 45%. According to one embodiment, phosphorous retention is increased by at least about 50%. According to one embodiment, phosphorous retention is increased by at least about 55%. According to one embodiment, phosphorous retention is increased by at least about 60%. According to one embodiment, phosphorous retention is increased by at least about 65%.

According to one embodiment, the pig is a sow. According to one embodiment, the sow is in a gestation period.

According to one embodiment, the at least one vitamin D compound is a vitamin D2 or D3 compound. According to one embodiment, the at least one vitamin D compound is a vitamin D3 compound. According to one embodiment, the vitamin D3 compound is 1-alpha-hydroxycholecalciferol.

According to one embodiment, the vitamin D composition is concurrently administered with a calcium and phosphorus source. According to one embodiment, the calcium and phosphorus source is dispersed in a feed, feed supplement or a combination thereof. According to one embodiment, the feed, feed supplement or a combination thereof comprises corn and one or more minerals. According to one embodiment, the minerals are selected from the group consisting of vitamin A, vitamin E, vitamin K, thiamin, riboflavin, pyridoxine, vitamin B12, panthothenic acid, niacin, folic acid, biotin, copper, iron, iodine, manganese, sodium selenium, and zinc. According to one embodiment, the vitamin D compound is a vitamin D3 compound.

A composition for increasing the apparent total tract digestibility and retention of calcium and phosphorus in a pig is provided. The composition includes an effective amount of a vitamin D composition comprising at least one vitamin D compound. The vitamin D composition as provided herein may further include one or more inert compounds or carriers that aid in administration. The vitamin D composition as provided herein may be formulated as a liquid, gel, powder, table, capsule, or any other acceptable formulation suitable for administration to pigs. Upon administration of the vitamin D composition to a pig, the apparent total tract digestibility and retention of calcium and phosphorus is increased.

According to one embodiment, the at least one vitamin D compound is a vitamin D2 or D3 compound. According to one embodiment, the at least one vitamin D compound is a vitamin D3 compound. According to one embodiment, the vitamin D3 compound is 1-alpha-hydroxycholecalciferol. According to one embodiment, the vitamin D3 compound is an air-stable, high-melt 1α-hydroxy-vitamin D₃ compound (alfacalcidol-1α,3β,5Z,7E-9,10-secocholesta-5,7,10(19)-triene-1,3-diol—illustrated below), crystalline hydrates, solvates, polymorphs and pharmaceutically acceptable salts thereof. According to one embodiment, the vitamin D3 compound has a melting point of about 140° C. to about 144° C. According to one embodiment, the vitamin D3 compound is characterized by a melt onset at about 141° C. According to one embodiment, the vitamin D3 compound is present substantially as a single polymorph.

According to one embodiment, the vitamin D3 compound as provided herein is prepared according to a process as provided in U.S. Ser. No. 16/286,880, the contents of which are incorporated herein by reference. According to one embodiment, the vitamin D3 compound as provided herein is prepared according to a process that includes the following steps:

treating vitamin D3 with sulfur dioxide to produce two cyclic compounds, each protected via a silicon-protecting group;

rearranging the silicon-protecting group compounds with sulfur dioxide extrusion via thermal isomerization to yield a silicon-protected 5,6-trans-vitamin D₃;

oxidizing 5,6-trans-vitamin D₃ via allylic oxidation to yield a 1α-hydroxy derivative;

de-protecting the 1α-hydroxy derivative to yield crystalline 1α-hydroxy-5,6-trans-vitamin D₃; and

photochemically isomerizng the crystalline 1α-hydroxy-5,6-trans-vitamin D₃ to yield 1α-hydroxy-vitamin D₃, wherein the 1α-hydroxy-vitamin D₃ has a melting point of about 140° C. to about 144° C. The method may further include the step of purifying the 1α-hydroxy-vitamin D₃ via polish filtration and recrystallizing the 1α-hydroxy-vitamin D₃ via a solvent exchange with at least one solvent selected from the group consisting of n-heptane, heptanes, and a combination thereof. The method of preparing the vitamin D3 compound may further include the step of purifying the 1α-hydroxy-vitamin D₃ via polish filtration and recrystallizing the 1α-hydroxy-vitamin D₃ via a solvent exchange with at least one solvent selected from the group consisting of n-heptane, heptanes, and a combination thereof.

According to one embodiment, the vitamin D composition is concurrently administered with a calcium and phosphorus source. According to one embodiment, the calcium and phosphorus source is dispersed in a feed, feed supplement or a combination thereof. According to one embodiment, the feed, feed supplement or a combination thereof comprises corn and one or more minerals. According to one embodiment, the minerals are selected from the group consisting of vitamin A, vitamin E, vitamin K, thiamin, riboflavin, pyridoxine, vitamin B12, panthothenic acid, niacin, folic acid, biotin, copper, iron, iodine, manganese, sodium selenium, and zinc.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

### Example 1

Testing was conducted to show that the calcium and phosphorus levels in diets fed to late gestating sows and supplementation of 1-alpha-hydroxycholecalciferol (1-α-OH-D3) affect apparent total tract digestibility (ATTD) and retention of calcium and phosphorus. The results indicate there is not an interaction between dietary calcium and phosphorus levels and supplementation of 1-α-OH-D3 in diet fed to gestating sows.

Thirty six gestating sows were allotted to three blocks of twelve sows using a randomized complete block design. Four diets were fed to the twelve sows in each block. Thus, there were a total of nine replicate sows per diet. Sows were fed experimental diets from day 91 to 105 of gestation and were housed individually in metabolism crates during this period. Metabolism crates were equipped with a feeder, a nipple drinker, and fully slatted T-bar floors. A screen floor and a urine pan were installed below the T-bar floors to allow for collection of feces and urine, respectively. The initial five days of each period in the metabolism crates were considered the adaptation period to the diets followed by four days of fecal collection using the marker to marker procedure (Adeola, O. 2001. Digestion and balance techniques in pigs. In: A. J. Lewis and L. L. Southern, editors, Swine Nutrition. CRC Press, Washington, D.C., USA. p. 903-916.). Fecal collection was initiated when the first marker (i.e., indigo carmine) appeared in the feces and ceased when the second marker (i.e., chromic oxide) appeared (Id.). Urine was collected in buckets placed under the urine pans with 50 mL of 3N HCl from day six in the morning until day ten in the morning. Buckets were emptied daily, the weight of the collected urine was recorded, and 10% of the collected urine was stored at −20° C. until subsampling.

Diets were formulated using a 2×2 factorial arrangement with two levels of calcium and phosphorus (i.e., 100% and 75% of the requirement; NRC, 2012) with a constant calcium to phosphorus ratio (See Table 1) without or with supplemental 1-α-OH-D3 (Alpha D3, Premex Inc., NC). The high level of calcium and phosphorus was 100% requirement for late gestation sows. The low level of calcium phosphorus was 75% of requirement for late gestation sows (NRC. 2012. Nutrient requirements of swine. 11th rev. ed. Natl. Acad. Press, Washington, D.C., USA). The corn-1-α-OH-D3 premix provided 12.5 mg Alpha D3 per kg of complete diet; 1,000 g of 1-α-OH-D3 premix was prepared by mixing 417 mg Alpha D3 and 999.583 g ground corn.

The vitamin-micromineral premix provided the following quantities of vitamins and micro minerals per kilogram of complete diet: vitamin A as retinyl acetate, 10,622 IU; vitamin D3 as cholecalciferol, 1,660 IU; vitamin E as selenium yeast, 66 IU; vitamin K as menadione nicotinamide bisulfate, 1.40 mg; thiamin as thiamine mononitrate, 1.08 mg; riboflavin, 6.49 mg; pyridoxine as pyridoxine hydrochloride, 0.98 mg; vitamin B12, 0.03 mg; D-pantothenic acid as D-calcium pantothenate, 23.2 mg; niacin, 43.4 mg; folic acid, 1.56 mg; biotin, 0.44 mg; Cu, 20 mg as copper chloride; Fe, 123 mg as iron sulfate; I, 1.24 mg as ethylenediamine dihydriodide; Mn, 59.4 mg as manganese hydroxychloride; Se, 0.27 mg as sodium selenite and selenium yeast; and Zn, 124.7 mg as zinc hydroxychloride.

TABLE 1

| | Item | | | |
|---|---|---|---|---|
| | High | | Low | |
| | Ca and P levels | | Ca and P levels | |
| 1-α-OH-D₃, mg/kg diet | 0 | 12.5 | 0 | 12.5 |
| Ingredients | | | | |
| Corn (%) | 72.91 | 69.91 | 73.73 | 70.73 |
| Soybean meal (%) | 14.00 | 14.00 | 14.00 | 14.00 |
| Sugar beet pulp (%) | 8.00 | 8.00 | 8.00 | 8.00 |
| Corn-vitamin D₃ premix | — | 3.00 | — | 3.00 |
| (%) | | | | |
| Calcium carbonate (%) | 1.01 | 1.01 | 0.83 | 0.83 |
| Monocalcium phosphate | 1.18 | 1.18 | 0.54 | 0.54 |
| (%) | | | | |
| Soybean oil (%) | 2.00 | 2.00 | 2.00 | 2.00 |
| Sodium chloride (%) | 0.40 | 0.40 | 0.40 | 0.40 |
| Vitamin-mineral premix | 0.50 | 0.50 | 0.50 | 0.50 |
| (%) | | | | |
| Analyzed nutrient | | | | |
| composition (%) | | | | |
| Gross Energy Intake | 3,831 | 3,848 | 3,827 | 3,861 |
| (kcal/kg) | | | | |
| Dry Matter (%) | 88.71 | 89.11 | 88.82 | 88.20 |
| Ash (%) | 4.82 | 4.48 | 3.97 | 3.99 |
| Crude Protein (%) | 12.64 | 12.10 | 12.10 | 12.41 |
| Ca (%) | 0.77 | 0.71 | 0.59 | 0.48 |
| P (%) | 0.53 | 0.49 | 0.38 | 0.37 |
| Phytate (%) | 0.74 | 0.69 | 0.69 | 0.65 |
| Phytate-P⁴ (%) | 0.21 | 0.19 | 0.19 | 0.18 |
| Non phytate-P (%) | 0.32 | 0.30 | 0.19 | 0.19 |
| Ca to P ratio | 1.46:1 | 1.43:1 | 1.56:1 | 1.31:1 |
| 1-α-OH-D₃ (μg/kg) | <0.64 | 4.96 | <0.64 | 3.46 |

All vitamins and minerals except calcium and phosphorus were included in all diets to meet or exceed nutrient requirements (NRC, 2012). Daily feed allotments were provided in a meal that was fed at 7 AM throughout the experiment. The daily feed allowance was 1.5 times the maintenance energy requirement for gestating sows based on the initial body weight of sows (i.e., 100 kcal ME/kg BW0.75; NRC. 2012. Nutrient requirements of swine. 11th rev. ed. Natl. Acad. Press, Washington, D.C., USA). Water was available at all times.

At the conclusion of the testing, urine samples were thawed and mixed within animal and collection period and subsamples were collected. Fecal samples were stored at −20° C. as soon as collected, and at the conclusion of the experiment, samples were dried at 65° C. in a forced air oven and finely ground through a 1-mm screen before analysis using a Wiley mill (Model 4; Thomas Scientific, Swedesboro, N.J.).

Concentration of 1-α-OH-D3 in the diets were analyzed using liquid chromatography with tandem mass spectrometry (Aronov, P. A., L. M. Hall, K. Dettmer, C. B. Stephensen, and B. D. Hammock. 2008. Metabolic profiling of major vitamin D metabolites using Diels-Alder derivatization and ultra-performance liquid chromatography-tandem mass spectrometry. Analytical and Bioanalytical Chemistry. 391:1917-1930. doi:10.1007/s00216-008-2095-8). Calcium and phosphorus in diets, feces, and urine samples were analyzed by inductively coupled plasma spectroscopy (AOAC Int. 2007. Official methods of analysis of AOAC int. 18th ed. Rev. 2nd ed. AOAC Int., Gaithersburg, Md., USA; method 985.01 A, B, and C) after wet ash sample preparation (AOAC Int., 2007; method 975.03 B(b)]).

Diets were analyzed for phytic acid (Ellis, R., E. R. Morris, and C. Philpot. 1977. Quantitative determination of phytate in presence of high inorganic-phosphate. Anal. Biochem. 77:536-539. doi:10.1016/0003-2697(77)90269-X). All diets and fecal samples were analyzed for dry matter (AOAC Int., 2007; method 930.15) and ash was measured in all diets (AOAC Int., 2007; method 942.05). Diet and fecal samples were analyzed for gross energy (Model 6400, Parr Instruments, Moline, Ill.) and crude protein was calculated as N×6.25 and N was analyzed by combustion (AOAC Int., 2007; method 990.03) using a LECO FP628 apparatus (LECO Corp., Saint Joseph, Mich.).

Normality of data and homogeneity were verified using the UNIVARIATE and MIXED procedure (SAS Inst. Inc., Cary, N.C.) and outliers were identified using Internally Studentized Residuals (Tukey, 1977). Sow was the experimental unit for all analyses. Data were analyzed using MIXED procedures of SAS (SAS Institute Inc., Cary, N.C.). The statistical model included level of calcium and phosphorus, supplemental 1-α-OH-D3, and the interaction as fixed effects and block, body weight group within block, and parity as random effects. Least squares means were calculated using the LSMeans statement in SAS and means separated using the PDIFF statement with Tukey's adjustment if the interaction was significant. Statistical significance and tendency were considered at P<0.05 and 0.05≤P<0.10, respectively.

No interactions between calcium and phosphorus levels and use of supplemental 1-α-OH-D3 were observed for feed intake, fecal and urine excretion, or calcium balance (See Table 2).

TABLE 2

| | High | | Low | | | | | |
|---|---|---|---|---|---|---|---|---|
| Item | Ca and P | | Ca and P | | | P-value | | |
| 1-α-OH-D₃, | Levels | | Levels | | | Ca and P | Vit | |
| (mg/kg diet) | 0 | 12.5 | 0 | 12.5 | SEM | Levels | D₃ | Interaction |
| Feed intake | 2.98 | 2.93 | 2.99 | 2.92 | 0.05 | 0.997 | 0.223 | 0.901 |
| (kilograms/day) | | | | | | | | |
| Fecal excretion | 0.39 | 0.32 | 0.35 | 0.30 | 0.06 | 0.110 | 0.002 | 0.464 |
| (kg dry matter/d) | | | | | | | | |
| Urine excretion | 10.51 | 11.26 | 12.10 | 13.91 | 3.40 | 0.327 | 0.554 | 0.804 |
| (kilograms/day) | | | | | | | | |
| ATTD of Dry Matter (%) | 85.04 | 87.72 | 86.80 | 88.31 | 2.17 | 0.076 | 0.003 | 0.365 |
| Ca balance | | | | | | | | |
| Ca intake (grams/day) | 22.00 | 21.61 | 15.90 | 15.55 | 0.28 | <0.001 | 0.194 | 0.929 |
| Fecal Ca output | 19.44 | 15.00 | 13.04 | 10.89 | 2.25 | <0.001 | 0.001 | 0.184 |
| (grams/day) | | | | | | | | |
| Absorbed Ca | 2.55 | 6.63 | 2.82 | 4.66 | 2.27 | 0.331 | 0.002 | 0.206 |
| (grams/day) | | | | | | | | |
| ATTD of Ca (%) | 11.31 | 30.25 | 18.21 | 30.49 | 12.17 | 0.407 | 0.001 | 0.439 |
| Urine Ca output | 0.32 | 0.59 | 0.44 | 0.99 | 0.13 | 0.020 | 0.001 | 0.201 |
| (grams/day) | | | | | | | | |
| Ca retention | 2.23 | 6.02 | 2.37 | 3.67 | 2.24 | 0.212 | 0.007 | 0.161 |
| (grams/day) | | | | | | | | |
| Ca retention | 9.80 | 27.48 | 15.41 | 24.07 | 11.97 | 0.796 | 0.005 | 0.292 |
| (% of intake) | | | | | | | | |

For Table 2, each least squares mean for each treatment represents nine observations, respectively, except for the two diets containing high or low calcium and phosphorous levels with no supplemental 1-α-OH-D3 (n=8). The high level of calcium and phosphorus was equal to the 100% requirement for late gestation sows. The low level of calcium and phosphorus was equal to 75% of requirement for late gestation sows (NRC, 2012).

Feed intake, fecal excretion, urine excretion, and the ATTD of dry matter by sows were not affected by the level of calcium and phosphorus in the diets. Calcium intake and fecal calcium output were greater (P<0.001) in sows fed high level of calcium and phosphorus compared with sows fed diets containing low calcium and phosphorus, but absorbed calcium and the ATTD of calcium was not affected by dietary calcium and phosphorus. Urine calcium output was less (P<0.05) if sows were fed diets containing more calcium and phosphorus compared with sows fed diets containing lower calcium and phosphorus, but dietary calcium and phosphorus levels did not affect calcium retention by sows.

Although feed intake was not different among treatments, fecal excretion was less (P<0.05) from sows fed diets supplemented with 1-α-OH-D3 compared with sows fed diets with no 1-α-OH-D3, which resulted in greater ATTD of dry matter in sows fed diets supplemented with 1-α-OH-D3 compared with sow fed no supplemental 1-α-OH-D3. Fecal calcium output was less (P<0.01), resulting in greater (P<0.01) absorbed calcium and ATTD of calcium in sows fed diets supplemented with 1-α-OH-D3 compared with sows fed no supplemental 1-α-OH-D3. Supplementation of 1-α-OH-D3 increased (P<0.01) urine calcium outputs from sows, but calcium retention was greater (P<0.01) in sows fed diets with supplemental 1-α-OH-D3 compared with sows fed no supplemental 1-α-OH-D3, because of the increase in absorbed calcium.

No interactions between calcium and phosphorus levels and use of supplemental 1-α-OH-D3 were observed in phosphorus balance except for fecal and urine phosphorus outputs (Table 3).

TABLE 3

| | High | | Low | | | | | |
|---|---|---|---|---|---|---|---|---|
| Item | Ca and P | | Ca and P | | | P-value | | |
| 1-α-OH-D₃ | levels | | levels | | | Ca and P | Vit | |
| (mg/kg diet) | 0 | 12.5 | 0 | 12.5 | SEM | Levels | D₃ | Interaction |
| P intake (grams/day) | 15.18 | 14.9 | 11.07 | 10.83 | 0.19 | <0.001 | 0.192 | 0.923 |
| Fecal P output (grams/day) | 12.77 | 9.71 | 8.52 | 7.67 | 1.44 | <0.001 | 0.001 | 0.049 |
| Absorbed P (grams/day) | 2.41 | 5.19 | 2.53 | 3.16 | 1.47 | 0.103 | 0.005 | 0.066 |
| ATTD of P (%) | 15.57 | 34.7 | 23.38 | 29.63 | 11.16 | 0.731 | 0.003 | 0.114 |
| Urine P output (grams/day) | 0.48 | 0.99 | 0.16 | 0.12 | 0.09 | <0.001 | 0.001 | <0.001 |
| P retention (grams/day) | 1.93 | 4.20 | 2.37 | 3.04 | 1.52 | 0.541 | 0.016 | 0.176 |
| P retention (% of intake) | 12.44 | 28.03 | 21.93 | 28.51 | 11.52 | 0.223 | 0.010 | 0.269 |

For Table 3, each least squares mean for each treatment represents nine observations, respectively, except for the two diets containing high or low Ca and P levels with no supplemental 1-α-OH-D3 (n=8). The high level of calcium and phosphorus was equal to the 100% requirement for late gestation sows. The low level of calcium and phosphorus was equal to 75% of requirement for late gestation sows (NRC, 2012).

Supplementation of 1-α-OH-D3 decreased (P<0.05) fecal and urine P output from sows fed diet containing high level of dietary calcium and phosphorus, but there was no difference between the two diets with low level of dietary calcium and phosphorus (interaction; P<0.05).

Phosphorus intake and fecal phosphorus output were greater (P<0.001) in sows fed high level of calcium and phosphorus compared with sows fed diets containing low calcium and phosphorus, but absorbed phosphorus and the ATTD of phosphorus was not affected by dietary calcium and phosphorus. Likewise, dietary calcium and phosphorus levels did not affect phosphorus retention by sows.

Regardless of dietary calcium and phosphorus levels, absorbed phosphorus, ATTD of phosphorus, and phosphorus retention were greater (P<0.05) in sows fed diets supplemented with 1-α-OH-D3 compared with sows fed no supplemental 1-α-OH-D3.

No interactions were observed in gross energy intake of pigs, ATTD of gross energy, and concentration of digestible energy in diets fed to sows (Table 4). There was no effect of calcium and phosphorus level, but the ATTD of gross energy and concentration of digestibility energy were increased (P<0.01) by supplementing 1-α-OH-D3 to diets fed to sows.

TABLE 4

| | High | | Low | | | | | |
|---|---|---|---|---|---|---|---|---|
| Item | Ca and P | | Ca and P | | | P-value | | |
| 1-α-OH-D₃ | Levels | | Levels | | | Ca and P | Vit | |
| (mg/kg diet) | 0 | 12.5 | 0 | 12.5 | SEM | Levels | D₃ | Interaction |
| Gross Energy Intake | 11,418 | 11,267 | 11,364 | 11,287 | 186 | 0.923 | 0.514 | 0.832 |
| (kcal/day) | | | | | | | | |
| ATTD of gross energy | 84.6 | 87.3 | 85.6 | 87.5 | 2.3 | 0.291 | 0.001 | 0.654 |
| (%) | | | | | | | | |
| Digestible Energy | 3,240 | 3,361 | 3,275 | 3,388 | 90 | 0.227 | <0.001 | 0.871 |
| (kcal/kg) | | | | | | | | |
| Digestible Energy | 3,652 | 3,772 | 3,687 | 3,841 | 101 | 0.077 | <0.001 | 0.542 |
| (kcal/kg Dry Matter) | | | | | | | | |

For Table 4, each least squares mean for each treatment represents 9 observations, respectively, except for the diet containing high or low calcium and phosphorus levels with no supplemental 1-α-OH-D3 (n=8). The high level of calcium and phosphorus was equal to the 100% requirement for late gestation sows. The low level of calcium and phosphorus was equal to 75% of requirement for late gestation sows (NRC, 2012).

Values for the ATTD and retention of calcium and phosphorus were in agreement with previous values for gestating sows (Nyachoti, C. M., J. S. Sands, M. L. Connor, and O. Adeola. 2006. Effect of supplementing phytase to corn- or wheat-based gestation and lactation diets on nutrient digestibility and sow and litter performance; Jang, Y. D., M. D. Lindemann, E. van Heugten, R. D. Jones, B. G. Kim, C. V. Maxwell, and J. S. Radcliffe. 2014. Effects of phytase supplementation on reproductive performance, apparent total tract digestibility of Ca and P and bone characteristics in gestating and lactating sows. Rev. Colomb. Cienc. Pecu. 27:178-193; Lee, S. A., L. V. Lagos, C. L. Walk, and H. H. Stein. 2019. Basal endogenous loss, standardized total tract digestibility of calcium in calcium carbonate, and retention of calcium in gestating sows change during gestation, but microbial phytase reduces basal endogenous loss of calcium. J. Anim. Sci. 97:1712-1721). Values for the ATTD of gross energy and digestible energy in diets fed to sows were greater than calculated values from growing pig data (NRC, 2012), but this can be explained by greater digestibility of energy in gestating sows compared with growing pigs (Le Goff, G., and J. Noblet. 2001. Comparative total tract digestibility of dietary energy and nutrients in growing pigs and adult sows. J. Anim. Sci. 79:2418-2427; Casas, G. A., and H. H. Stein. 2017. Gestating sows have greater digestibility of energy in full fat rice bran and defatted rice bran than growing gilts regardless of level of feed intake. J. Anim. Sci. 95:3136-3142).

Results from this experiment demonstrated that the ATTD and retention of calcium and phosphorus were increased by 1-α-OH-D3 supplementation. Absorption of calcium by the active transport is regulated by calcitriol, which is the active form of vitamin D (Crenshaw, T. D. 2001. Calcium, phosphorus, vitamin D, and vitamin K in swine nutrition. In: A. J. Lewis and L. L. Southern, editors, Swine Nutrition. 2nd ed. CRC Press, Boca Raton, Fla., USA. p. 187-212). It is possible that supplementation of 1-α-OH-D3 may upregulate the absorption of calcium in the intestinal tract and resorption of calcium in the kidney of sows because 1-α-OH-D3 is an active vitamin D3 analog.

The increases in the ATTD of calcium and phosphorus were also driven by the reduced fecal dry matter excretion, which resulted in an increase in the ATTD of dry matter in this experiment. There was also increases in the ATTD of gross energy and digestible energy with supplementation of 1-α-OH-D3, which concurred with data for the ATTD of dry matter. The increase in the ATTD of dry matter and gross energy was unexpected.

There were no data demonstrating the effects of decreasing calcium and phosphorus on digestibility and retention of calcium and phosphorus in gestating sows. Previous data demonstrated that the ATTD of calcium and phosphorus were not affected by increasing calcium and phosphorus intake by sows (Lee, S. A., G. A. Casas, and H. H. Stein. 2018. The level of feed intake does not influence digestibility of calcium and phosphorus in diets fed to gestating sows, but gestating sows have reduced digestibility of calcium and phosphorus compared with growing gilts. Can. J. Anim. Sci. 98:591-594) and this was in agreement with the results from this experiment.

Calcium and phosphorus balance by sows in late gestation were not affected by dietary calcium and phosphorus levels, but supplementation of 1-α-OH-D3 increased the ATTD and retention of calcium and phosphorus and the ATTD of gross energy and concentration of digestible energy in diets fed to sows regardless of dietary calcium and phosphorus.

We claim:

1. A method of increasing apparent total tract digestibility and retention of calcium and phosphorus in a pig, the method comprising the step of:
administering to the pig an effective amount of a vitamin D composition comprising at least one vitamin D compound,
wherein upon administration to a pig, the apparent total tract digestibility and retention of calcium and phosphorus is increased.

2. The method of claim 1, wherein the vitamin D composition is concurrently administered with a calcium and phosphorus source.

3. The method of claim 2, wherein the calcium and phosphorus source is dispersed in a feed, feed supplement or a combination thereof.

4. The method of claim 3, wherein the feed, feed supplement or a combination thereof comprises corn and one or more minerals.

5. The method of claim 4, wherein the minerals are selected from the group consisting of vitamin A, vitamin E, vitamin K, thiamin, riboflavin, pyridoxine, vitamin B12, panthothenic acid, niacin, folic acid, biotin, copper, iron, iodine, manganese, sodium selenium, and zinc.

6. The method of claim 1, wherein the vitamin D compound is a vitamin D3 compound.

7. The method of claim 6, wherein the vitamin D3 compound is 1-alpha-hydroxycholecalciferol.

8. The method of claim 1, wherein calcium digestibility is increased by at least about 10%.

9. The method of claim 1, wherein phosphorus digestibility is increased by at least about 5%.

10. The method of claim 1, wherein calcium retention is increased by at least about 110%.

11. The method of claim 1, wherein phosphorous retention is increased by at least about 65%.

12. The method of claim 1, wherein the pig is a sow.

13. The method of claim 12, wherein the sow is in a gestation period.

12. A composition for increasing the apparent total tract digestibility and retention of calcium and phosphorus in a pig, the composition comprising:
an effective amount of a vitamin D composition comprising at least one vitamin D compound,
wherein upon administration to a pig, the apparent total tract digestibility and retention of calcium and phosphorus is increased.

13. The composition of claim 12, wherein the vitamin D composition is concurrently administered with a calcium and phosphorus source.

14. The composition of claim 12, wherein the calcium and phosphorus source is dispersed in a feed, feed supplement or a combination thereof.

15. The composition of claim 14, wherein the feed, feed supplement or a combination thereof comprises corn and one or more minerals.

16. The composition of claim 14, wherein the minerals are selected from the group consisting of vitamin A, vitamin E, vitamin K, thiamin, riboflavin, pyridoxine, vitamin B12, panthothenic acid, niacin, folic acid, biotin, copper, iron, iodine, manganese, sodium selenium, and zinc.

17. The composition of claim 12, wherein the vitamin D compound is a vitamin D3 compound.

18. The composition of claim 17, wherein the vitamin D3 compound is 1-alpha-hydroxycholecalciferol.

19. The composition of claim 12, wherein calcium digestibility is increased by at least about 10%.

20. The composition of claim 12, wherein phosphorus digestibility is increased by at least about 5%.

21. The composition of claim 12, wherein calcium retention is increased by at least about 110%.

22. The composition of claim 12, wherein phosphorous retention is increased by at least about 65%.

23. The composition of claim 12, wherein the pig is a sow.

24. The composition of claim 23, wherein the sow is in a gestation period.
